(19) United States
(12) Patent Application Publication
(10) Pub. No.: US 2005/0256458 A1
(43) Pub. Date: Nov. 17, 2005

(54) Self-anchoring catheter and method for using same

(75) Inventors: Pamela Howard, Allentown, PA (US); Eben Howard, Allentown, PA (US); Steven C. Walker, Waxahachie, TX (US); John M. Shepherd, San Antonio, TX (US)

Correspondence Address: CAHN & SAMUELS LLP, 2000 P STREET NW, SUITE 200, WASHINGTON, DC, 20036, US

(73) Assignee: Eben Howard and Pamela A. Howard, Allentown, PA (US)

(21) Appl. No.: 11/108,131

(22) Filed: Apr. 18, 2005

Related U.S. Application Data
(60) Provisional application No. 60/563,006, filed on Apr. 19, 2004.

(51) Int. Cl. A61M 29/00

(52) U.S. Cl. 604/174; 604/104

(57) ABSTRACT

A self-securing catheter to prevent dislodgement from skin or tissue preferably includes a hub and a cannula with at least one anchoring mechanism extending from the hub and/or the cannula. In some embodiments, the anchoring mechanisms can be flexible to allow them to protrude outwardly from the catheter. The catheter can be easily removed from the patient's tissue or skin by a medical professional with a twisting motion (e.g., clockwise or counterclockwise rotation of the catheter).

This application claims the benefit of U.S. provisional application Ser. No. 60/563,006, filed on Apr. 19, 2005, which is incorporated herein by reference.

### I. FIELD OF THE INVENTION

The present invention generally relates to catheters. More specifically, the present invention relates to the securing of catheters to a patient's tissue or skin.

### II. BACKGROUND OF THE INVENTION

A variety of devices can be employed in human and veterinary medicine to deliver medication to a particular site in an attempt to benefit a patient. For example, a medical catheter can be used to deliver medication to a patient's brain or spinal cord in an attempt to treat a malady, disease, or illness. Devices can also be employed to administer fluids or nutrients to a patient. For example, a medical IV catheter can be used to deliver fluids through the skin of a dehydrated patient. In addition to the above uses, various devices can also be inserted into a patient to withdraw blood and to measure pressures within the blood vessel. Regardless of the employed device, however, it must be securely attached to a patient, either internally (for example, into organ tissue) or to the skin. Regardless of the reason for inserting the device, maintaining controlled placement of the device in the position of interest is highly desirable. In some instances, the delivered drugs are highly concentrated but are more effective if they are precisely delivered to the position of interest.

Further, in some instances, only a small portion of a highly concentrated drug is required to be therapeutically beneficial to the patient. Thus, failure to maintain controlled placement of the device in the position of interest (that is, dislodgement of the device) may be detrimental to the patient by causing her/him to receive more than the required amount of the highly concentrated drug.

In addition to the previously described side effects of dislodgement, accidental migration to an undesired location can cause bleeding, infection, collapse of a lung, heart rhythm abnormalities, and other potentially fatal complications. Regardless of whether such complications ensue, such accidental migration or dislodgment requires repositioning or replacing the catheter, which can waste invaluable time and can subject the patient to further risks and possibly additional trauma and punctures.

Several methods have been used in attempts to secure catheters more reliably. One method commonly employed to immobilize a catheter is that of simply using sections of adhesive tape to secure the catheter such as a medical IV catheter to the skin of a patient. The taping method has obvious drawbacks. For example, it is cumbersome to use and remove the tape. Further, the tape often does not move with the compliance modulus of the skin. Thus, it is difficult to use this method with a patient who is restless.

Another method employs a pair of plastic wings with each wing having an eyelet. The catheter is inserted into the patient, up to the hub. The medical professional then passes a needle and thread through one of the eyelets and then through the patient's skin. Finally, the medical professional ties a knot, cuts the thread, and repeats the process on the opposite eyelet. The sewing technique is not entirely reliable, as the wings often break, especially if tension is applied to cinch the catheter. The pressure exerted on the skin is highly variable and suturer dependent. Too little pressure may result in a loose, floppy attachment that allows the catheter to slide in and out of its insertion site, with the dangerous consequences described above. Too much pressure, however, may cause skin necrosis and breakdown, which may cause a persistent ulcer, infection, and/or the undesirable effects of dislodgement.

Moreover, suturing is also tedious and time-consuming. Many small parts such as the needle and thread must be handled. This can increase the chances that one of these parts will be dropped off the sterile field and contaminated. Straight needles are generally employed for the suturing process because they eliminate the cost of a curved needle and the instrument to hold it (a needle driver). As a result, however, the skin must be awkwardly pinched, and the suturing process itself is more traumatic.

Finally, there is also a risk of penetrating too deeply with the needle and puncturing a vital structure. These problems occur when attempting to attach a catheter to a patient who is not motionless. When the patient is unable or unwilling to remain motionless long enough for the catheter to be attached, this procedure becomes even more difficult and prone to error.

Notwithstanding the usefulness of the existing catheter securing devices, a need exists for a more dependable and safer catheter-securing device that can be employed to easily secure a catheter to a patient and prevent dislodgement of the catheter, or more particularly, a medical IV catheter.

### III. SUMMARY OF THE INVENTION

The present invention addresses the need for a more dependable and safer apparatus and method for securing a catheter assembly. The self-securing catheter assembly of the present invention can be employed to secure a catheter such as a medical IV, for example, to a patient to prevent dislodgement of the catheter.

In at least one embodiment, the catheter assembly includes a hub with a longitudinal axis, a distal portion, a proximal portion, and an axial bore. In addition to the hub, the catheter assembly includes a cannula having at least one bore extending from the distal portion of the hub. In this embodiment, the cannula includes anchoring mechanisms that allow the catheter assembly to be secured to the patient, thereby preventing dislodgement. The anchoring mechanisms can be also located on the hub of the catheter assembly. In at least one embodiment, the anchoring mechanisms can be flexible and can protrude outwardly from the catheter.

In at least one embodiment, the self-securing catheter assembly of the present invention includes a tubular body and at least one anchoring mechanism located on the tubular body for allowing the catheter to be anchored to the patient. Regardless of the featured embodiment, the catheter assembly is inserted into a patient's tissue or skin. The anchoring mechanisms are then lodged within the patient's skin, thereby securing the catheter assembly to the patient's tissue or skin to prevent catheter dislodgement.

In at least one embodiment, the invention includes a catheter having a hub, a cannula extending from the hub, and a plurality of anchors extending from the cannula towards the hub.

In at least one embodiment, the invention includes a catheter capable of being anchored to a patient having a tubular body, and at least one anchoring mechanism located on the tubular body for allowing the catheter to be anchored to the patient.

In at least one embodiment, the invention includes a catheter having a hub having a distal portion and a proximal portion, the hub having a passageway passing therethrough, the hub having at least one anchor extending from the distal portion, and a cannula extending from the distal portion of the hub, and the cannula having a passageway passing therethrough in communication with the passageway of the hub.

In at least some of the embodiments of the present invention, the catheter assembly can be easily removed from the patient's tissue or skin by a medical professional with a twisting motion (for example, clockwise or counterclockwise rotation of the catheter assembly). During such rotation, the anchoring mechanisms located on the cannula and/or hub are bent to allow easy removal of the catheter assembly. In some embodiments of the present invention, the anchoring mechanisms may be bent and wound around the catheter assembly during the rotation.

An object of at least one embodiment the present invention is to prevent dislodgement of a catheter from tissue or skin.

Another object of at least one embodiment of the present invention is to increase the dependability of the attachment of catheters to tissue or skin.

Another object of at least one embodiment of the present invention is to minimize the difficulty in the process of securing catheters to tissue or skin.

An advantage of at least one embodiment of the present invention is decreased time involved in securing the catheter to tissue or skin.

Given the following enabling description of the drawings, the apparatus and method of the present invention should become evident to a person of ordinary skill in the art.

### IV. BRIEF DESCRIPTION OF THE DRAWINGS

Like reference numerals in the figures represent and refer to the same element or function. The use of cross-hatching within these drawings should not be interpreted as a limitation on the potential materials used for construction of the invention.

FIG. 1 illustrates a view of an embodiment of the present invention depicting a catheter assembly in an anchored state.

FIGS. 2A and 2B illustrate views of embodiments of the present invention depicting a catheter assembly in an anchored state in which anchoring mechanisms are located on the cannula.

FIG. 3 illustrates a view of an embodiment of the present invention depicting a catheter assembly in an unanchored state.

FIG. 4 illustrates a top view of an embodiment of the present invention depicting a catheter in an anchored state.

FIG. 5 illustrates a top view of an embodiment of the present invention depicting a catheter in an unanchored state.

### V. DETAILED DESCRIPTION OF THE INVENTION

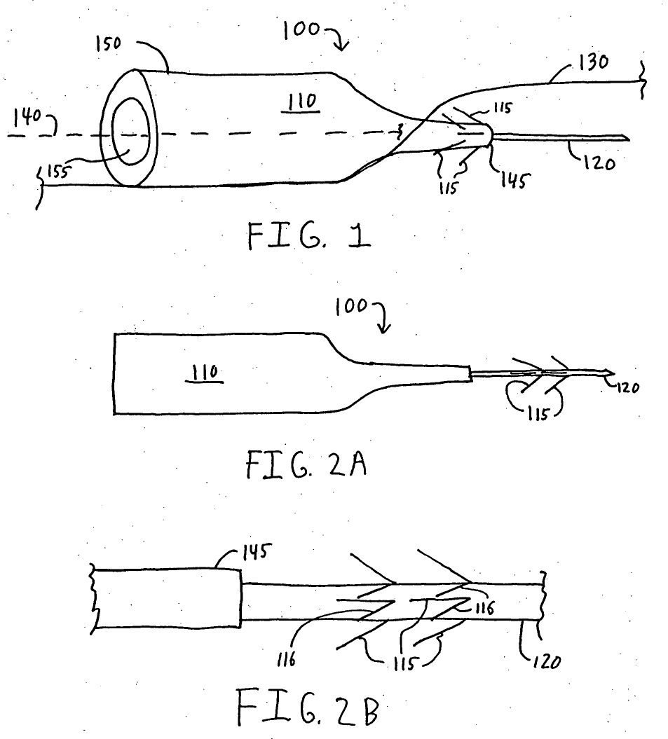

FIGS. 1-3 illustrate exemplary embodiments of the invention directed to a self-anchoring catheter (or tubular body) 100 for securing or anchoring the catheter 100 to a patient's tissue or skin. The catheter 100 is self-anchoring in that it can be inserted into a patient's tissue or skin 130 (shown in FIG. 1) and can secure itself to the patient's tissue or skin 130 to prevent dislodgement.

FIG. 1 illustrates an exemplary embodiment of the catheter 100 having a hub 110 and a cannula 120, which together form a tubular body. In the exemplary embodiment depicted in FIG. 1, the hub 110 includes a longitudinal axis 140, a distal portion 145, and a proximal portion 150. The distal portion 145 includes anchoring mechanisms (or anchors) 115. The anchors 115 collectively are a means for anchoring the catheter to the patient. The cannula 120 extends from the distal portion 145 of the hub 110. The hub 110 and the cannula 120 have a bore (or passageway) 155 passing therethrough from the proximal portion 150 to the free end (or insertion tip) of the cannula 120.

The catheter 100 can be lodged within the tissue or skin 130 (shown in FIG. 1) to allow the anchoring mechanisms 115 to penetrate the tissue or skin 130, thereby causing the catheter 100 to enter an anchored state, as illustrated, for example, in FIGS. 1, 2A, and 4. For example in the exemplary embodiment of FIG. 1, the hub 110 is preferably inserted approximately one-half centimeter underneath the patient's skin. It should be noted, however, that the depth at which the hub 110 is inserted will depend on a particular patient and the thickness of the patient's skin.

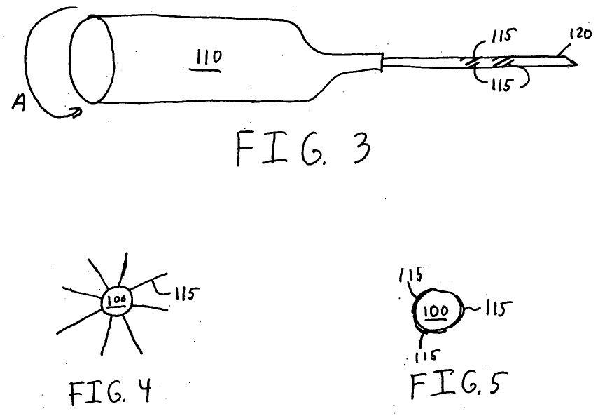

FIGS. 3 and 5 illustrate catheter 100 in an unanchored state in which the anchoring mechanisms 115 are shown bent toward the hub 110 and/or the cannula 120. The anchoring mechanisms 115 in at least one embodiment when in the unanchored state reside in channels 116 present in the hub 110 and/or cannula 120, for example in FIG. 2B.

Although the hub 110 appears to have a bottle shape in the exemplary embodiments shown in the figures, it should be noted that the hub 110 may have the form of any other viable shape. For example, the hub may also have a conical shape.

In embodiments of the present invention, the anchoring mechanisms 115 protrude outwardly from the catheter 100 in an anchored state, as shown in FIGS. 1, 2A and 4. Such protrusions can be more effective in allowing the catheter 100 to remain lodged into the tissue or skin 130. It should be noted that the anchoring mechanisms 115 can be any means for fixing, anchoring, or securing the catheter 100 to the tissue or skin 130. Further, as illustrated in FIGS. 3 and 5, the anchoring mechanisms 115 preferably are made of flexible material to allow them to bend to allow removal of the catheter 100 from the tissue or skin 130. By rotating the catheter 100 in a clockwise or counterclockwise manner, the state of the catheter 100 can be altered from an anchored state shown in FIGS. 1, 2A, and 4 to an unanchored state shown in FIGS. 3 and 5. The catheter 100 in at least one embodiment is in an anchored state upon insertion. Alternatively, the anchoring mechanism 115 may be unwound into the tissue or skin 130 for anchoring of the catheter 100 after insertion.

FIG. 2 illustrates an exemplary embodiment with the anchoring mechanisms 115 located along the cannula 120. Alternatively, the anchoring mechanisms 115 may also be located on hub 110 in addition to the cannula 120.

Examples of the anchoring mechanisms 115 include the following: a hook, a spike, a barb, a needle, or any other anchoring or securing mechanism viable for securing the catheter 100 to the tissue or skin 130 after the catheter 100 penetrates the tissue or skin 130. It should be noted that the anchoring mechanisms 115 may be included at various locations on the catheter 100 and may include any number of anchors 115 as the number and arrangements illustrated in the figures are for exemplary purposes only.

In embodiments of the present invention, the anchoring mechanisms 115 can be manufactured of dissolvable material such as vicryl or “cat gut.” For example, each of the anchoring mechanisms 115 depicted in FIGS. 1-5 can be made of dissolvable material such that it eventually dissolves after being lodged in the tissue or skin 120. Depending upon the intended use, the anchoring mechanisms 115 can be made from any harden material, e.g., metal particularly where the securest attachment is desired during a medical evacuation from the battlefield during which the patient is likely to be tossed about.

The embodiments of the present invention described herein allow the device (for example, a catheter) to be secured to the patient's tissue or skin with ease and prevent the catheter from becoming dislodged from the patient's tissue or skin. For the reasons described above, the prevention of such dislodgement from the patient is of the utmost importance.

The exemplary and alternative embodiments described above may be combined in a variety of ways with each other. Furthermore, the steps and number of the various steps illustrated in the Figures may be adjusted from that shown.

As used above “substantially,” “generally,” and other words of degree are relative modifiers intended to indicate permissible variation from the characteristic so modified. It is not intended to be limited to the absolute value or characteristic which it modifies but rather possessing more of the physical or functional characteristic than its opposite, and preferably, approaching or approximating such a physical or functional characteristic.

Although the present invention has been described in terms of particular embodiments, it is not limited to those embodiments. Alternative embodiments, examples, and modifications which would still be encompassed by the invention may be made by those skilled in the art, particularly in light of the foregoing teachings.

Those skilled in the art will appreciate that various adaptations and modifications of the embodiments described above can be configured without departing from the scope and spirit of the invention. Therefore, it is to be understood that, within the scope of the appended claims, the invention may be practiced other than as specifically described herein.

1. A catheter comprising:
a hub having a distal portion and a proximal portion,
said hub having a passageway passing therethrough,
said hub having at least one anchor extending from the distal portion, and
a cannula extending from said distal portion of said hub, and said cannula having a passageway passing therethrough in communication with the passageway of said hub.

2. The catheter of claim 1, wherein said cannula is capable of removal by rotating said catheter to bend said at least one anchor to unsecure said hub from a patient.

3. A catheter capable of being anchored to a patient, comprising:
a tubular body; and
at least one anchoring mechanism located on said tubular body for allowing the catheter to be anchored to the patient.

4. The catheter of claim 3, wherein said anchoring mechanism is capable of dissolving after a period of time.

5. The catheter of claim 3, wherein said anchoring mechanism includes flexible material.

6. The catheter of claim 3, wherein the tubular body is capable of being rotated to secure it to tissue.

7. The catheter of claim 3, wherein the tubular body is capable of being rotated to unsecure it from tissue.

8. The catheter of claim 8, wherein said anchoring mechanism is capable of being wound around the tubular body during said rotation.

9. The catheter of claim 3, wherein said anchoring mechanism includes a hook.

10. The catheter of claim 3, wherein said at least one anchoring mechanism includes a spike.

11. The catheter of claim 3, wherein said at least one anchoring mechanism includes a barb.

12. The catheter of claim 3, wherein said anchoring mechanism includes a needle.

13. The catheter of claim 3, wherein said at least one anchoring mechanism is manufactured of vicryl.

14. A catheter comprising:
a hub,
a cannula extending from said hub, and
a plurality of anchors extending from said cannula towards said hub.

15. The catheter of claim 14, wherein said anchors include a spike.

16. The catheter of claim 14, wherein said anchors include a barb.

17. The catheter of claim 14, wherein said cannula includes a channel for each anchor, each channel running along the outside of said cannula from said anchor.

18. The catheter of claim 14, wherein said cannula includes a channel for each anchor, each channel running along the outside of said cannula from said anchor for a distance equal to a length of said anchor.

19. A method for securing the catheter of claim 14 to tissue, the method comprising:
providing the catheter of claim 14;
inserting the cannula into tissue; and
rotating the cannula to cause the anchors to contact the tissue to secure the cannula to the tissue.

20. The method of claim 19, further comprising:
rotating the cannula to cause the anchors to wrap around the cannula to allow removal of the cannula from the tissue.
